(19) United States
(12) Patent Application Publication
(10) Pub. No.: US 2023/0165486 A1
(43) Pub. Date: Jun. 1, 2023

(54) DYNAMO TORQUE ANALYZER

(71) Applicant: Texas Tech University System, Lubbock, TX (US)

(72) Inventors: Ty Palmer, Palmer, TX (US); Jarrod Blinch, Lubbock, TX (US)

(73) Assignee: Texas Tech University System, Lubbock, TX (US)

(21) Appl. No.: 17/801,775

(22) PCT Filed: Feb. 25, 2021
(86) PCT No.: PCT/US2021/019749

§ 371 Date: Aug. 23, 2022

Related U.S. Application Data
(60) Provisional application No. 62/982,046, filed on Feb. 26, 2020.

(51) Int. Cl. A61B 5/11; A61B 5/00

(52) U.S. Cl. CPC A61B 5/1107; A61B 5/725; A61B 5/7221; A61B 5/7225; A61B 2560/0214; A61B 2560/0223; A61B 2562/0252

(57) ABSTRACT

A method of analyzing a subject using a dynamo torque analyzer includes recording the force produced during an isometric contraction of the subject with a load cell, processing data associated with the isometric contraction, and calculating and displaying, via a microcomputer of the dynamo torque analyzer, peak torque, and rate of torque development values using the data associated with the isometric contraction. The dynamo torque analyzer calculates and displays the peak torque and rate of torque development values in real time.

### CROSS-REFERENCES TO RELATED APPLICATIONS

This Application claims priority to U.S. Provisional Pat. Application No. 62/982,046, filed on Feb. 26, 2020. The entirety of the aforementioned application is incorporated herein by reference as if fully set forth herein.

### TECHNICAL FIELD OF THE INVENTION

The present invention relates in general to the field of peak torque and rate of torque development, and specifically to an analyzer for determining peak torque and rate of torque development.

### BACKGROUND

Muscle strength characteristics, such as peak torque (PT) and rate of torque development (RTD), are commonly measured to assess athletic ability, identify functional deficiencies, and monitor neuromuscular performance changes in response to training or fatigue-related interventions. The torque signal recorded during an isometric maximal voluntary contraction (MVC) is often used to measure PT and RTD variables. Research suggests that isometric PT is an important parameter for strength-based performances and movement activities such as walking and stairclimbing. However, because isometric PT requires greater than 300 ms to be achieved, it may not be as functionally relevant for explosive-type activities, such as sprinting or jumping, which require rapid muscle response times of less than 250 ms.

Alternatively, rapid torque characteristics, which include RTD, have functional significance in fast and forceful muscle contractions and thus, may be better predictors of explosive performances. Research has reported that isometric RTD was a more effective variable than PT of the leg extensor and flexor muscles at explaining the variance associated with vertical jump power in young and older adults. Moreover, it has been shown that isometric leg extension RTD was better than PT at discriminating between soccer players with different sprinting, agility, and vertical jump performance abilities. Taken together, these findings provide support that isometric leg extension and flexion muscle strength, and in particular RTD, may play an important role in the ability to perform explosive-type movements, both in highly trained athletes and in older adults.

Previous studies investigating isometric PT and RTD characteristics have measured these variables using laboratory-based isokinetic dynamometers. Isokinetic dynamometers are reliable devices for measuring isometric PT and RTD; nevertheless, the implementation of these devices in an applied setting may be hindered by their high cost and lack of portability. Other researchers have measured isometric PT and RTD characteristics using portable hand-held transducers. However, previous studies investigating the validity of PT and RTD measurements from portable transducers have reported conflicting results. Moreover, isokinetic dynamometers and hand-held transducers do not provide real-time calculations of isometric RTD. These calculations require extensive offline analysis with data processing software. The cost, time, and expertise required for offline analysis are all barriers to the successful calculation of RTD in laboratory and field-based settings.

In older adults, preservation of walking ability is essential for maintaining functional independence. However, numerous studies investigating walking ability as a function of age have reported reduced walking performances in older populations. A common measure used to assess walking performance in older adults is the distance covered during a six-minute walk test. Previous studies in older adults have reported mean six-minute walk distances ranging from 401 to 714 m. Because ~550 m is considered the maximal walking distance required for important activities of daily living (i.e., grocery shopping, going to department stores, etc.), the ability to walk this distance in six minutes may be used as a criterion to determine functional status in older adults. Decreased six-minute walk distance (<550 m) has been associated with an increased incidence of functional limitations, which may lead to an elevated risk of future disability and mortality. Thus, the prospect of identifying performance-based measures that can successfully distinguish between older adults who are able versus those who are unable to walk 550 m in six minutes may be critical in the assessment and treatment of functional decline in aging populations.

Maximal and rapid strength measurements, such as PT and RTD, have been shown to be significant predictors of walking ability and thus, could be potentially useful for distinguishing between functional performance capacities in older adults. The torque signal produced during an isometric MVC is often used to measure PT and RTD variables. Previous studies have demonstrated that isometric PT and RTD of the knee extensor muscles are effective parameters at discriminating between older adults of different functional performance abilities. However, because the performance measures used in these studies were limited to 10-m gait speed and timed up-and-go data, it remains unclear whether PT and RTD can effectively distinguish between functional differences in older adults according to their performance on a six-minute walk test. The six-minute walk test is different than other mobility assessments (i.e., 10-m gait speed, timed up-and-go test, etc.) in that it requires sustained walking over an extended period of time. Nevertheless, despite being a longer duration event, the six-minute walk test still requires a certain level of muscle strength and power and therefore, may also be influenced by maximal and rapid strength characteristics. Research suggests that older adults with lower maximal and rapid strength use a greater percentage of their force-generating capacity to support and propel the body forward during walking. Operating at a greater capacity contributes to the early onset of fatigue, which may reduce one’s ability to walk a long distance within a specific duration of time. Thus, in light of this and given the potential importance of maximal and rapid strength to locomotor function, it is possible that isometric PT and RTD may be effective variables at determining six-minute walk distance in older adults.

Many locomotor-related movement tasks, including walking, require force application response times of less than 200 ms. Consequently, RTD assessed over a short time period (0-200 ms) may be more functionally relevant and thus, a better discriminator of locomotor performances than PT, which often requires more than 300 ms to be achieved. A number of studies have presented evidence in support of this hypothesis by demonstrating the superior capacity of RTD versus PT to distinguish between individuals of different gait speed and timed up-and-go performance abilities. In addition, previous studies have shown that RTD but not PT of the knee extensors was significantly associated with 10-m gait speed and 30-s sit-to-stand performances in older populations. Although these findings reveal the capacity of RTD to predict functional performances assessed over a short distance (10 m) or duration (30 s), it has yet to be determined if RTD is an effective predictor of functional performance for a longer duration event, such as the six-minute walk test. Further research is needed to examine the relationships between six-minute walk distance and PT and RTD characteristics in older adults.

### SUMMARY

A dynamo torque analyzer (or simply referred to herein as “dynamo”) includes a load cell that can be affixed to existing equipment found in laboratories and clinics (i.e., isokinetic dynamometer, resistance training machine, treatment table or chair, boots, ankle braces, etc.) and a microcomputer that records torque in newton-meters (Nm). Unlike previous transducers, the dynamo automatically calculates and displays real-time measurements of PT and RTD immediately after an isometric contraction. The dynamo is portable, easy-to-use, and offers a time and cost-effective means for quantifying isometric PT and RTD.

In some aspects, a method of analyzing a subject using a dynamo torque analyzer includes recording, via the dynamo torque analyzer, the force produced during an isometric contraction of the subject, processing, via the dynamo torque analyzer, the torque signal associated with the isometric contraction, and calculating and displaying, via the dynamo torque analyzer, measurements of peak torque and rate of torque development using the torque signal associated with the isometric contraction. The dynamo torque analyzer includes a microcomputer comprising a CPU and memory, and a load cell coupled to the microcomputer.

In some aspects, a dynamo torque analyzer includes a microcomputer comprising a CPU and memory, a display in communication with the microcomputer, a load cell in communication with the microcomputer, and a power supply coupled to the microcomputer.

### BRIEF DESCRIPTION OF THE DRAWINGS

For a more complete understanding of the present invention and for further objects and advantages thereof, reference may now be had to the following description taken in conjunction with the accompanying drawings in which:

FIG. 1 is a perspective view illustrating a dynamo torque analyzer according to aspects of the disclosure;

FIG. 2 is a schematic illustrating a dynamo torque analyzer according to aspects of the disclosure;

FIG. 3 is a perspective view of a load cell of a dynamo torque analyzer according to aspects of the disclosure;

FIG. 4 illustrates a first configuration for testing PT and RTD according to aspects of the disclosure;

FIG. 5 illustrates a second configuration for testing PT and RTD according to aspects of the disclosure;

FIG. 6 illustrates a method of testing for PT and RTD according to aspects of the disclosure;

FIGS. 7A-7D illustrate relationships between a Biodex isokinetic dynamometer and a dynamo torque analyzer for isometric leg extension and flexion PT, Peak RTD, RTD 100, and RTD200, respectively, according to aspects of the disclosure;

FIGS. 8A-8D illustrate Bland-Altman plots for assessing agreement between a Biodex isokinetic dynamometer and a dynamo torque analyzer for isometric leg extension and flexion PT, Peak RTD, RTD100, and RTD200, respectively, according to aspects of the disclosure;

FIG. 9 is a graph of a processed torque signal derived from a dynamo torque analyzer for a trial according to aspects of the disclosure;

FIGS. 10A-10D illustrate relationships between six-minute walk distance and isometric knee extension PT, Peak RTD, RTD100, and RTD200, respectively, for a dynamo torque analyzer according to aspects of the disclosure; and

FIG. 11 is a graph illustrating countermovement according to aspects of the disclosure.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

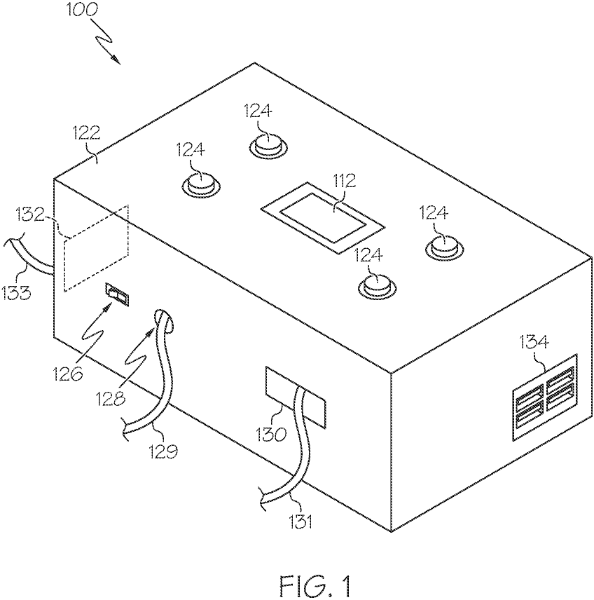

FIG. 1 is a perspective view of a dynamo torque analyzer 100 according to aspects of the disclosure. A housing 122 houses components of dynamo 100 and provides a mounting point for various interface components of dynamo 100. A display 112 and interface controls 124 are positioned on a top side of housing 122. Display 112 displays menu items, prompts, messages, and data (e.g., PT, RTD values, and other values). Interface controls 124 permit a user to operate dynamo 100. Various tasks such as running a trial on a patient to measure, for example, PT and values for RTD, may be initiated via interface controls 124. Interface controls 124 are illustrated as buttons in FIG. 1, those of skill in the art will appreciate that that interface controls 124 may include other types of interactive controls such as rotary knobs, toggles, switches, touch pads, touch screens, keyboards, mice, and the like.

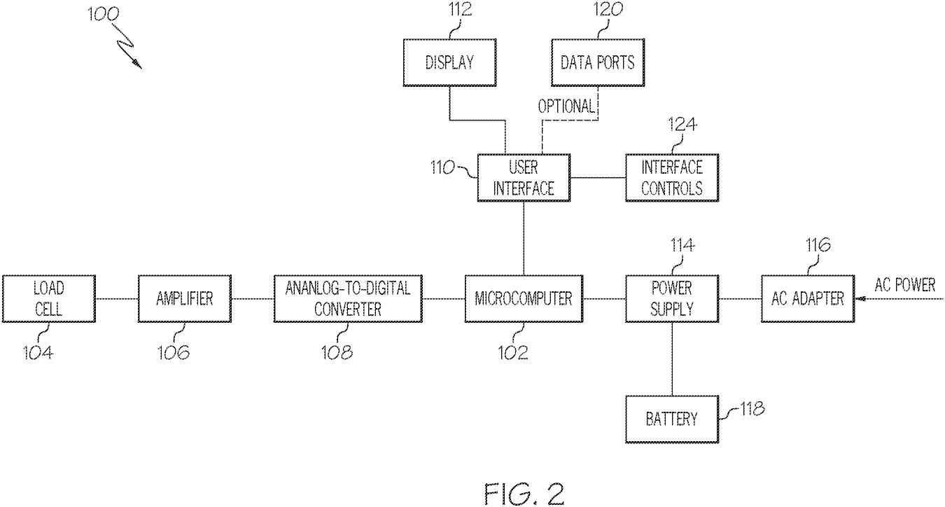

A front side of housing 122 includes a power switch 126, a pass through 128, and a pass through 130. Sides of housing 122 include a pass through 132 and a pass through 134. Power switch 126 permits dynamo 100 to be powered off and on. Pass through 128 provides an opening to route one or more cables, such as a power cable 129, into housing 122. Power cable 129 may be connected to an external power supply to change a battery within housing 122 or to simply power dynamo 100. Pass through 130 provides an opening to route one or more cables, such as an external display cable 131, into housing 122. External display cable 131 connects dynamo 100 to an external display, such as a monitor, to display, for example, the information on display 112 and/or additional information and data generated by dynamo 100. Pass through 132 provides an opening for load cell cable 133 to pass into housing 122. Load cell cable 133 couples a load cell (see 104 in FIG. 2) to dynamo 100. Pass through 134 provides an opening for data ports (see 120 in FIG. 2) associated with a microcomputer (see 102 in FIG. 2) of dynamo 100. The data ports permit peripherals (e.g., mouse, keyboard, and the like) and/or data storage devices (e.g., storage media) to be connected to dynamo 100.

Those having skill in the art will recognize that components of dynamo 100 can be rearranged as desired. In some aspects, display 112 and/or interface controls 124 can be moved to other faces of housing 122. In some aspects, pass throughs 128-134 can be moved to other faces of housing 122. In some aspects, two or more of pass throughs 128-134 can be combined and/or relocated and components that passed through separate openings can jointly pass through a single opening.

FIG. 2 is a schematic illustrating dynamo torque analyzer 100 according to aspects of the disclosure. Dynamo 100 includes a microcomputer 102 (e.g., a single-board computer comprising a CPU and memory capable of executing instructions). Microcomputer 102 may be any of various types of computing devices, such as a Raspberry Pi 3 Model B+. Microcomputer 102 is configured with software to receive inputs such as a signal from a load cell 104, inputs from a user via interface controls 124, and inputs/outputs from data ports 120 (e.g., communications with peripheral devices and storage devices). Data ports 120 may comprise one or more of USB ports, serial ports, and the like. Microcomputer 102 includes storage media (e.g., flash media or the like) to store executable instructions and/or data. A user interface 110, which comprises a collection of wires, passes communications between microcomputer 102, display 112, data ports 120, and interface controls 124.

Load cell 104 is configured to measure a force input upon it by a subject (e.g., a patient or participant). Operation of load cell 104 will be discussed in more detail below. Various types of load cells may be used, such as an S-type load cell rated for 200 kg. Load cell 104 outputs a voltage signal in response to a force being applied to it. The voltage signal from load cell 104 is amplified by an amplifier 106 (e.g., a load cell/Wheatstone amplifier) and converted from an analog signal to a digital signal by analog-to-digital converter 108 (e.g., ADS1015 12-bit ADC). Analog-to-digital converter 108 then outputs a digital signal to microcomputer 102, which processes the signal and determines values, such as PT and various RTD values, from the processed signal. The processing of the signal and calculations are discussed in more detail below. A power supply 114 powers dynamo 100 and receives power from an AC adapter 116 that plugs into a power source and/or from a battery 118 (e.g., a rechargeable lithium polymer battery). AC adapter 116 receives AC power from, for example, a wall outlet to power dynamo 100 and/or recharge battery 118. Battery 118 may be recharged and provides power to dynamo 100 so that dynamo 100 may be temporarily operated without being plugged into an external power supply.

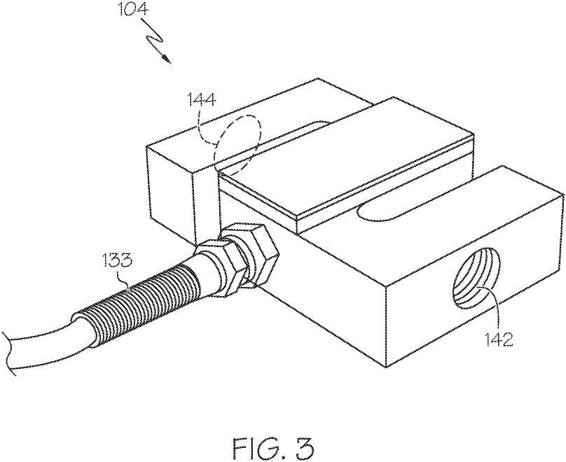

FIG. 3 is a perspective view of load cell 104 according to aspects of the disclosure. Load cell 104 is illustrated as an S-type load cell and includes a pair of mounting points 142, 144 (illustrated in FIG. 3 as threaded bores, but could be other types of mounting points such as hooks, loops, latches, and the like) that allow load cell 104 to connect to a wide variety of testing apparatuses etc. Load cell cable 133 connects load cell 104 to amplifier 106.

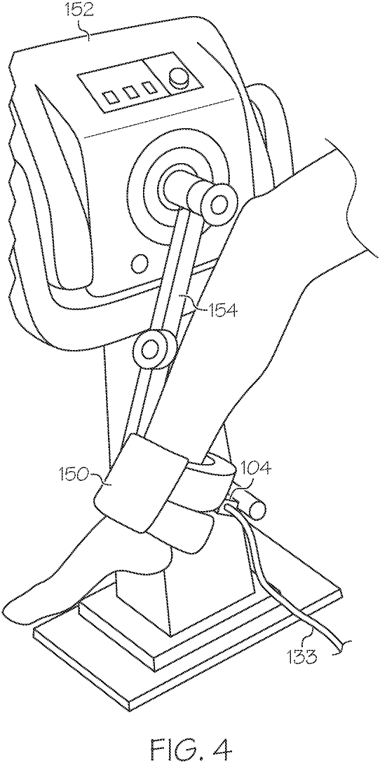

FIG. 4 illustrates a first configuration for testing PT and RTD according to aspects of the disclosure. In the first configuration, load cell 104 is attached via mounting point 142 to an ankle cuff 150 and via mounting point 144 to an isokinetic dynamometer 152. Ankle cuff 150 provides a comfortable and secure way for load cell 104 to be attached to an ankle of a patient. In some aspects a boot (e.g., a therapy boot) or pad may be used in place of ankle cuff 150. As shown in FIG. 4, the patient’s point of rotation (i.e., knee) is aligned with a rotational axis of an arm 154 of isokinetic dynamometer 152. Force output by a user is imparted to load cell 104 and isokinetic dynamometer 152 and recorded. Load cell cable 133 transmits a signal (e.g., a voltage signal) from load cell 104 to amplifier 106. Connecting load cell 104 to isokinetic dynamometer 152 allows for data to be simultaneously collected from load cell 104 and isokinetic dynamometer 152, which permits data from load cell 104 to be verified against isokinetic dynamometer 152.

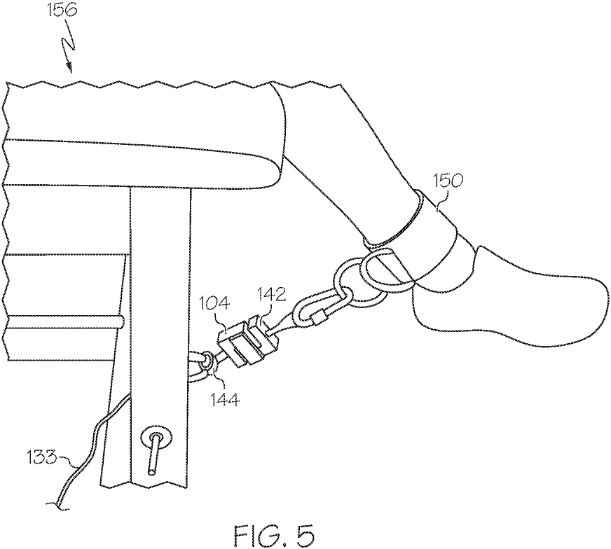

FIG. 5 illustrates a second configuration for testing PT and RTD according to aspects of the disclosure. The second configuration differs from the first configuration of FIG. 4 in that load cell 104 is used without an isokinetic dynamometer and is instead connected between ankle cuff 150 and a table 156 via mounting points 142, 144, respectively. In other aspects, table 156 could be a different anchoring point, such as a wall, chair, weight, and the like.

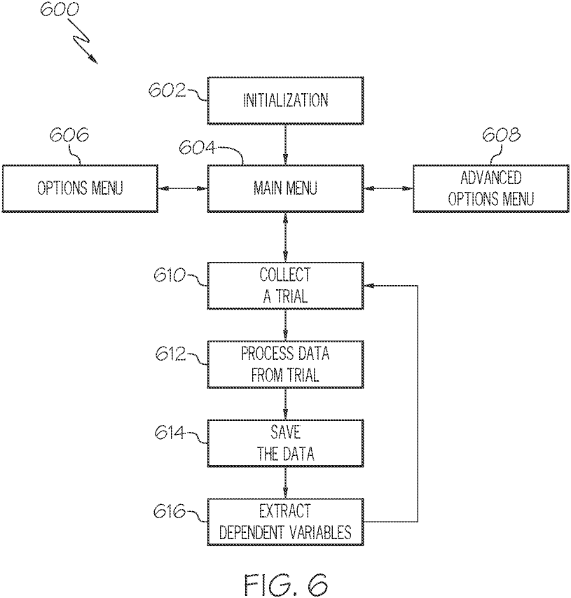

FIG. 6 illustrates a method 600 for testing for PT and RTD according to aspects of the disclosure. Method 600 begins with an initialization step 602. At step 602, microcomputer 102 initializes dynamo 100 for operation. Initialization includes loading settings from the options and advanced options that were previously configured. In some aspects, dynamo 100 is configured with a standard or default operating configuration so that no additional configuration is needed unless a non-standard operating mode is desired. In some aspects, the input/output of dynamo 100 may be configured in step 602. After step 602, method 600 proceeds to step 604.

In step 604, display 112 displays several selectable options: an options menu, an advanced options menu, and a command to begin a trial. If the options menu is selected (e.g., by a user), method 600 proceeds to step 606. In the options menu, a user may set a subject number, which is a unique identifier for a subject, and a limb length. Limb length is a measure of the length of the subject’s limb (e.g., from a subject’s joint center, such as a knee, to load cell 104). After step 606, method 600 returns to step 604. If the advanced options menu is selected (e.g., by a user), method 600 proceeds to step 608. In the advanced options menu, a user may select a baseline check, set calibration parameters of dynamo 100 to convert a voltage output from load cell 104 to newtons, set a cutoff frequency, and set a contraction onset. These options are discussed in more detail below. After step 608, method 600 returns to step 604. If the option to begin a trial is selected (e.g., by a user), method 600 proceeds to step 610.

In step 610, microcomputer 102 executes commands to collect a trial. A trial is the performance of a single isometric contraction by the subject. An isometric contraction describes a contraction where a muscle tenses but does not change length. To collect a trial, the subject’s limb, for example a leg, is connected to load cell 104 (e.g., see FIGS. 4 and 5). When prompted by display 112, the subject performs an isometric contraction that is collected by dynamo 100. The collection of a trial is discussed in more detail below. After a trial is collected, method 600 proceeds to step 612.

In step 612, data collected from the trial is processed by microcomputer 102 prior to determining PT and RTD. The processing may include using a dual-pass Butterworth filter, converting a voltage output from the load cell to newtons using calibration parameters consisting of a slope (m) and intercept (b) value that are inputted into dynamo 100 in step 608, converting newtons to newton-meters, correcting for gravity, and interpolating the signal from the load cell to 1,000 Hz. The processing of the data is discussed in more detail below. After the data is processed, method 600 proceeds to step 614. In step 614, microcomputer 102 stores the raw and processed data for future reference. The data may be stored in memory/storage associated with microcomputer 102. After saving the raw and processed data, method 600 proceeds to step 616.

In step 616, microcomputer 102 analyzes the processed data and calculates desired values, such as PT and values for various RTD measurements. Analysis of the data may include checking for a steady baseline and identifying a contraction onset. After confirming a steady baseline and identifying the contraction onset, PT and various RTD values are calculated by microcomputer 102. If microcomputer 102 determines that there was not a steady baseline, dynamo 100 displays a message to the user that a new trial should be collected. The values for PT and RTDs are displayed on display 112 and/or saved to storage associated with microcomputer 102. Once values of PT and RTD have been determined, the values can be displayed on display 112 and/or saved to storage associated with microcomputer 102. After step 616, method 600 returns to step 610 to collect another trial or ends if no additional trials are desired.

### Methods

### Comparison Between Isokinetic Dynamometer and Dynamo

### Subjects

Twenty healthy young adults (10 male subjects, 10 female subjects; age = 22 ± 3 years; height = 169 ± 10 cm; mass = 71 ± 18 kg) volunteered to participate in this study. None of the subjects reported any current or ongoing neuromuscular diseases or musculoskeletal injuries specific to the ankle, knee, or hip joints. All subjects were considered as being recreationally active based on their self-reported levels of exercise behaviors (9 ± 4 h. wk⁻¹). Subjects were instructed to maintain the same lifestyle between sessions and to refrain from any vigorous physical activity or exercise within 24 h of testing. This study was approved by the university’s institutional review board for human subject research, and each subject was informed of the benefits and risks of the investigation before signing an informed consent document.

### Experimental Design

This study used a repeated-measures design to compare the reliability and magnitude of isometric leg extension and flexion PT and RTD measurements between an isokinetic dynamometer and the dynamo torque analyzer. Each subject visited the laboratory three times, separated by 2 - 7 days at approximately the same time of day (± 2 h). The first visit was a familiarization session and the next two visits were experimental sessions (session 1 and 2) from which data were collected and used for subsequent analyses. During the familiarization session, subjects practiced the testing procedures by performing several isometric MVC trials for the leg extensors and flexors. For each experimental session, subjects completed three isometric leg extension and flexion MVCs using an isokinetic dynamometer and the dynamo torque analyzer. Both of these devices were calibrated once at the start of the study.

To calibrate the dynamo, users can place known weights (i.e., 10, 25, and 45 lb. weight) onto the load cell. After placing a known weight onto the load cell, a trial is recorded. After the trial, a raw value appears on the display of the dynamo. This raw value is the arbitrary unit collected by the dynamo during the trial. The raw value is recorded. This process is repeated for each weight. After obtaining values for each weight, a program, such as a spreadsheet, can be used to determine slope-intercept values. After obtaining the slope (m) and intercept (b), these values are inputted into the dynamo under the calibration option.

### Isometric Torque

Torque signals were recorded simultaneously from a Biodex System 3 isokinetic dynamometer (Biodex Medical Systems Inc., Shirley, NY) and the dynamo torque analyzer during each MVC assessment. A configuration similar to that shown in FIG. 4 was used. The dynamo, for example dynamo torque analyzer 100, includes a microcomputer and a load cell (capacity: 200 kg) that was attached to the ankle cuff (or pad) of a lever arm (e.g., arm 154) fastened around the subject’s lower leg. The lever arm was connected to the input axis of the isokinetic dynamometer which was aligned with the axis of rotation of the knee joint. For each MVC, subjects sat on the dynamometer chair in an upright position with restraining straps placed over the shoulders, waist, and thigh. All MVCs were performed on the right leg at a knee joint angle of 60 ° below the horizontal plane. Prior to the MVC assessments, subjects performed a standardized warm-up of three submaximal isometric leg extension and flexion muscle actions at approximately 75% of their perceived maximal effort. Following the submaximal contractions, each subject performed three isometric MVCs of the leg extensors and flexors with one minute of recovery between each contraction and three minutes of recovery between muscle groups. The order of the leg extensor and flexor testing was randomized to control for any potential effects of fatigue. For all isometric MVCs, subjects were verbally instructed to “push” or “pull” “as hard and fast as possible” for a total of 3-4 s and strong verbal encouragement was given throughout the duration of the contraction.

### Data Processing

During each MVC, the torque signal from the isokinetic dynamometer was sampled at 1000 Hz with a Biopac data acquisition system (MP150WSW, Biopac System Inc., Santa Barbara, CA) and processed offline using custom-written software (Lab VIEW 11.0, National Instruments, Austin, TX). The scaled torque signal (Nm) was gravity corrected and low-pass filtered with a zero-phase lag, fourth-order Butterworth filter at a cutoff frequency of 150 Hz. Isometric PT was calculated as the highest mean 500 ms epoch. RTD was calculated as the linear slope of the torque signal (Δtorque/Δtime) at early and late time intervals of 0-100 (RTD 100) and 0-200 (RTD200) ms from contraction onset. Maximum RTD (Peak RTD) was calculated as the highest slope value for any 100 ms epoch that occurred over the initial 200 ms of the torque signal.

To calculate maximal and rapid torque characteristics using the dynamo, each subject’s limb length (distance in meters from the knee joint to the ankle) was entered into the dynamo’s microcomputer. The microcomputer multiplies the limb length of the subject by the force from the load cell to estimate torque. Force from the load cell was sampled and then interpolated to 1000 Hz, converted to torque, gravity corrected, and low-pass filtered with a 150 Hz cutoff frequency (zero-phase lag, fourth-order Butterworth filter). In a typical aspect, the contraction onset is set to a low value so as to permit early detection of a contraction, but not so low as to prematurely detect an onset as a result of noise in the signal. The contraction onset for the dynamo and isokinetic dynamometer was set at 1.0 Nm. This automated onset was used to ensure the same threshold value was being selected across all contractions and to allow the early portion of the MVC signal to be captured in the RTD output parameters. Isometric PT, Peak RTD, RTD 100, and RTD200 were calculated (using the same methods as those described above for the isokinetic dynamometer) and displayed by the microcomputer at the conclusion of each trial (see online supplementary video).

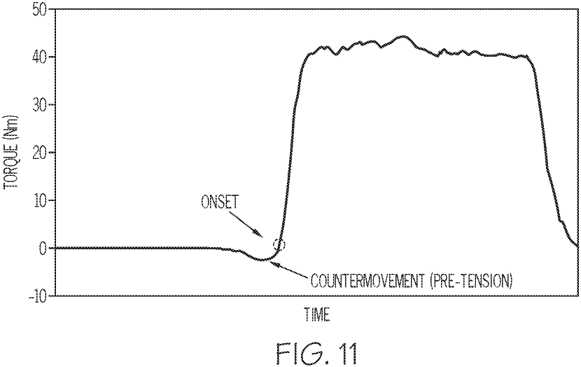

A unique feature of the dynamo is its ability to detect unsteady baseline torque. An unsteady baseline torque is caused by a countermovement (e.g., pretension) by the subject, which results in a bad reading for the baseline torque for a trial (see FIG. 11). Unsteady baseline torque resulting from either pre-tension or countermovement can adversely influence RTD (e.g., render the data inaccurate). The ability of the dynamo to alert the user about countermovement at the time of a trial enables another trial to be performed while the subject is still present. In the past, countermovements were not ascertained at the time of data collection. In some instances, getting the subject back for testing could be difficult. The dynamo’s microcomputer evaluated unsteady baseline torque by computing the baseline slope prior to contraction onset. If unsteady baseline torque was detected prior to contraction onset, a warning was displayed by the microcomputer at the end of the trial. Contractions with unsteady baseline torques, as indicated by the dynamo, were always discarded, and additional MVCs were performed until three leg extension and flexion contractions presented acceptable data. Of the three MVCs performed, the MVC with the highest Dynamo PT value during each session for leg extension and flexion was selected for further analysis.

### Statistical Analyses

Paired samples t-tests were used to examine the systematic variability in the PT and RTD characteristics of the leg extensors and flexors for the isokinetic dynamometer and Dynamo devices across sessions 1 and 2. The intraclass correlation coefficient (ICC) representing relative consistency (test-retest reliability) and the standard error of measurement (SEM), minimal difference (MD) needed to be considered real, and coefficient of variation (CV) representing absolute consistency were calculated for the leg extensors and flexors across sessions for each device. The ICC and SEM were also calculated for the leg extensors and flexors across trials within the familiarization session for the isokinetic dynamometer. Both the SEM and MD were expressed as absolute values and percentages of the mean. Differences in CV values between the isokinetic dynamometer and dynamo were analyzed by paired samples t-tests.

Model “2,1” from Shrout and Fleiss was used to calculate the ICC. Model 2,1 uses random and systematic error in the denominator of the ICC equation, and consequently, the ICCs generated with this model can be generalized to other laboratories and testers. The ICC (2,1) was calculated with the following equation:

$$I C {C}_{2 , 1} = \frac{M {S}_{S} - M {S}_{E}}{M {S}_{S} + \left(k - 1\right) M {S}_{E} + \left(k \left(M {S}_{T} - M {S}_{E}\right) / n\right)}$$

where MSs is the mean square for subjects, MS_{E} is the mean square error, MS_{T} is the mean square trials, k is the number of trials, and n is the sample size.

The SEM was calculated with the following equation:

$$S E M = \sqrt{M {S}_{E}}$$

The MD was calculated with the following equation:

$$M D = S E M \times 1.96 \times \sqrt{2}$$

The CV refers to the intra-subject variability of the measurements between sessions 1 and 2. For each subject, the CV was calculated using the following equation:

$$\text{CV} = \left(\frac{\text{SD of Measurements}}{\text{Mean of Measurements}}\right) \times 100$$

Differences in mean PT and RTD characteristics between the isokinetic dynamometer and dynamo from session 2 were analyzed by paired samples t-tests. Pearson product-moment correlation coefficients (r) and bias ± 95% limits of agreement (as represented by Bland-Altman plots) from session 2 data were used to assess the relationships between the isokinetic dynamometer and dynamo PT and RTD values. The calculations for the ICC, SEM, MD, and CV were performed using a custom-written spreadsheet (Microsoft Excel; Microsoft Corporation, Redmond, WA). All other statistical analyses were performed using SPSS software (version 26.0, IBM Corp, Armonk, NY). An alpha level of P ≤ 0.050 was considered statistically significant for all analyses.

### Results

There was no systematic variability (P = 0.105-0.994) across sessions for any of the leg extension and flexion PT and RTD characteristics for the isokinetic dynamometer and dynamo. Reliability analysis revealed that leg extension and flexion PT, Peak RTD, RTD 100, and RTD200 were highly consistent across sessions for both the isokinetic dynamometer and dynamo devices, with ICCs ranging between 0.935 and 0.984 and SEM% values between 4.93 and 14.46% (Table 1). There were no significant differences (P = 0.189-0.939) in CV values between devices for any of the variables except for leg flexion PT (P = 0.004). For this variable, the CV value for the Dynamo was slightly higher than that for the Biodex (Table 1).

TABLE 1

Reliability statistics across sessions for isometric leg extension and flexion peak torque and rate of torque development variables for the Biodex isokinetic dynamometer and dynamo.

| Leg Extension | | | | Leg Flexion | |
|---|---|---|---|---|---|
| Variable | Statistics | Biodex | Dynamo | Biodex | Dynamo |
| PT | P-value Δ[95% CI] ICC_{2,1} SEM (Nm) SEM% MD (Nm) MD% CV | 0.797 -0.9 [-8.3-6.4] 0.979 11.13 6.24 30.84 17.29 4.80 | 0.994 0.0 [-7.1-7.0] 0.982 10.68 5.94 29.59 16.45 5.03 | 0.215 -2.0 [-5.3-1.3] 0.984 4.93 4.93 13.67 13.68 4.54 | 0.193 -2.7 [-7.0-1.5] 0.976 6.42 6.38 17.80 17.69 6.18* |
| Peak | P-value | 0.451 | 0.424 | 0.105 | 0.144 |
| RTD | Δ[95% CI] ICC_{2,1} SEM (Nm·s⁻ SEM% MD (Nm·s-¹) MD% CV | -25.4 [-94.6-0.959 104.53 10.38 289.74 28.78 8.22 | -27.6 [-98.5-0.957 107.07 10.73 296.78 29.74 8.91 | 32.1 [-7.4-71.7] 0.942 59.75 10.59 165.62 29.36 9.31 | 30.8 [-11.5-0.935 63.91 11.41 177.15 31.64 10.37 |
| RTD 100 | P-value Δ[95% CI] ICC_{2,1} SEM (Nm·s⁻ SEM% MD (Nm·s⁻¹) | 0.834 -8.6 [-92.8-75.7] 0.938 127.35 14.46 353.01 | 0.976 1.2 [-83.4-85.9] 0.939 127.89 14.29 354.49 | 0.409 -13.3 [-46.4-0.952 49.95 11.69 138.45 | 0.770 -5.4 [-43.5-32.7] 0.936 57.59 13.62 159.63 |
| RTD200 | MD% CV P-value | 40.08 10.41 0.521 | 39.61 10.78 0.544 | 32.39 9.15 0.400 | 37.76 10.55 0.537 |
| | Δ[95% CI] ICC2.1 SEM (Nm·s⁻ SEM% MD (Nm·s-¹) MD% CV | -16.3 [-68.5-0.937 78.90 12.49 218.71 34.62 9.95 | -15.0 [-65.8-0.941 76.79 12.03 212.86 33.35 9.99 | 9.0 [-12.8-30.8] 0.960 32.95 8.68 91.32 24.07 7.71 | 7.6 [-17.7-33.0] 0.950 38.32 9.98 106.21 27.67 8.60 |

P-value - type I error rate for the paired samples t-test across sessions 1 and 2. Δ [95% CI] = within-device change across sessions [95% confidence interval]. ICC_{2,1} = intraclass correlation coefficient, model 2.1. SEM = standard error of measurement, expressed as absolute values and percentages of the mean. MD = minimal difference to be considered real, expressed as absolute values and percentages of the mean. CV = coefficient of variation. *Significant difference between devices (P ≤ 0.050).

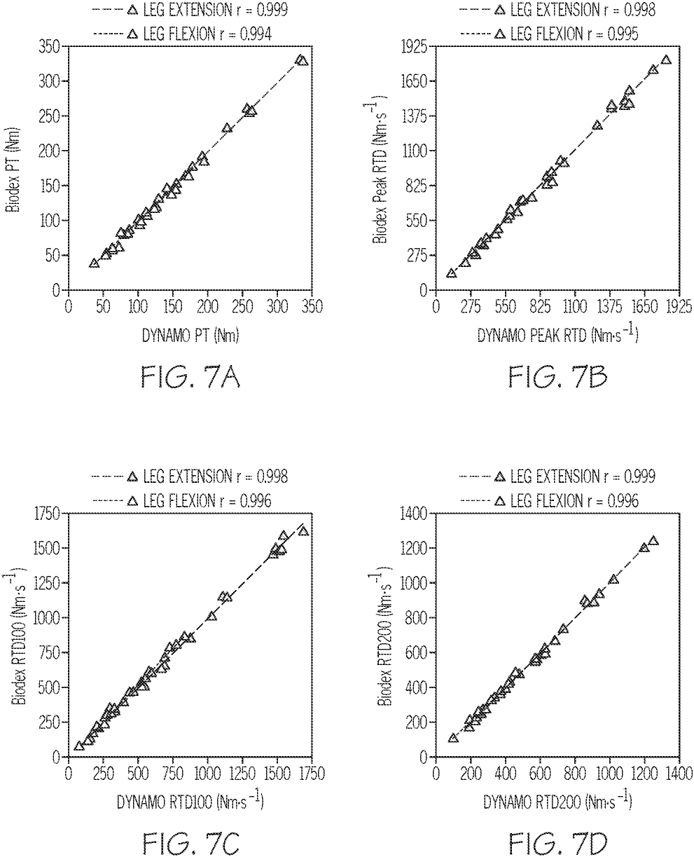

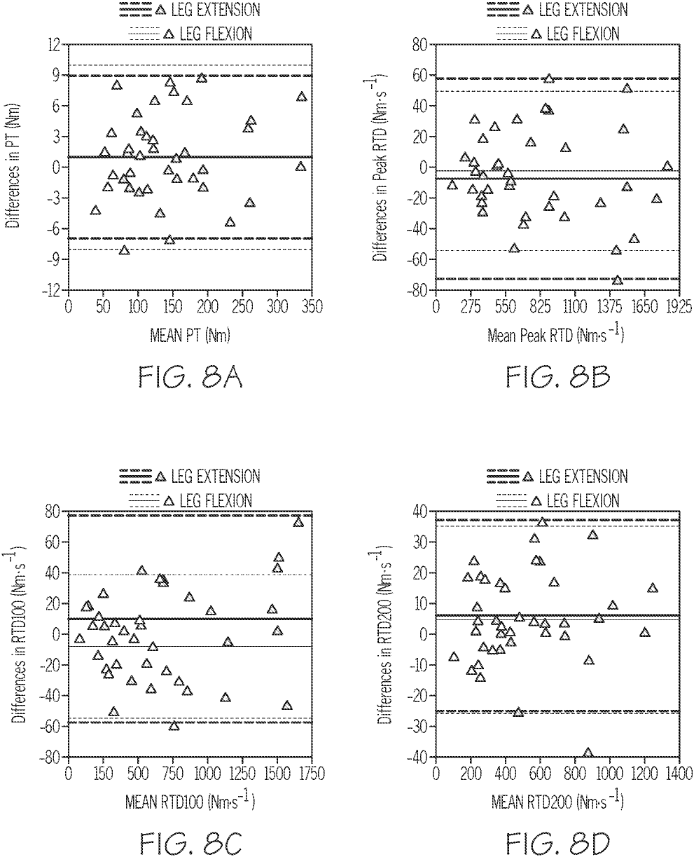

The within-session ICCs and SEM% values across trials for the isokinetic dynamometer ranged from 0.944-0.989 and 3.97-12.42%, respectively. There were significant positive relationships (r ≥ 0.994; P < 0.001) between the isokinetic dynamometer and Dynamo PT and RTD characteristics for the leg extensors and flexors (FIGS. 7A-7D). No significant differences (P = 0.107-0.555) between devices were observed for leg extension and flexion PT and RTD characteristics (Tables 2 and 3), and at least 95% of the differences between devices for these variables fell within the limits of agreement (FIGS. 8A-8D).

### Discussion

The high ICCs ≥0.935) and relatively low SEM% values (≤14.46%) observed in the present study across sessions for leg extension and flexion PT, Peak RTD, RTD100, and RTD200 demonstrated that the isokinetic dynamometer and Dynamo Torque Analyzer were both reliable assessment tools for measuring isometric maximal and rapid torque characteristics of the leg extensors and flexors from MVC assessments in healthy, recreationally-active young male and female subjects (Table 1).

These findings were comparable to the reliability of previous isometric MVC studies examining PT measurements of the leg extensors and flexors using isokinetic dynamometers in young adult populations, reporting ICCs of 0.880 - 0.975 and SEM% values of 4.20 - 8.80%. For rapid torque production, isometric leg extension and flexion reliability coefficients of 0.810 - 0.940 and SEM% values of 6.56 - 16.41% have been reported for Peak RTD, RTD 100, and RTD200, which are also comparable to those observed in the present study. An interesting finding of this study was the lack of differences in CV values between devices for all of the variables except for leg flexion PT (Table 1). For this variable, the CV value for the Dynamo was 6.18%, which was slightly higher than that for the Biodex (4.54%). Previous research has suggested that a variable with a CV value of 15% or less indicates acceptable reliability. Therefore, as the CV value for leg flexion PT on the Dynamo was less than 15%, we are confident that this variable is still reliable, and therefore, an acceptable parameter for assessing the maximal torque capabilities of the muscle.

In this study, the Dynamo provided measurements of PT and RTD that were strongly associated with (r = 0.994-0.999; FIGS. 7A-7D) and not significantly different than those of the isokinetic dynamometer (Tables 2 and 3). Moreover, the majority of the differences in the Bland-Altman plots were less than 10% of the mean values for each variable, suggesting good agreement between the two devices (FIGS. 8A-8D).

TABLE 2

Means (SD), P-values, bias (d) [95% CI], and limits of agreement (LOA) for isometric leg extension peak torque and rate of torque development variables for the Biodex isokinetic dynamometer and dynamo.
Leg Extension

| Variable | Biodex | Dynamo | P | d [95% CI] | LOA |
|---|---|---|---|---|---|
| PT (Nm) Peak RTD (Nm·s⁻¹) RTD 100 (Nm·s⁻¹) RTD200 (Nm·s⁻¹) | 178.9 (77.4) 1019.5 (517.7) 1 885.0 (500.7) 640.0 (321.6) | 179.8 (78.0) 1011.9 (513.4) 894.3 (508.7) 645.8 (320.6) | 0.281 0.308 0.229 0.107 | 0.9 [-0.9-2.8] -7.6 [-22.9-7.6] 9.3 [-6.4-25.0] 5.8 [-1.4-13.2] | -7.0-8.9 -73.0-57.7 -57.7-76.3 -25.2-37.0 |

TABLE 3

Means (SD), P-values, bias (d) [95% CI], and limits of agreement (LOA) for isometric leg flexion peak torque (PT) and rate of torque development (RTD) variables for the Biodex isokinetic dynamometer and dynamo.
Leg Flexion

| Variable | Biodex | Dynamo P | d [95% CI] | LOA |
|---|---|---|---|---|
| PT (Nm) Peak RTD (Nm·s⁻¹) RTD 100 (Nm·s⁻¹) RTD200 (Nm·s⁻¹) | 100.9 (40.2) 548.0 (261.4) 434.1 (243.4) 374.9 (164.6) | 102.0 (41.4) 0.307 544.6 (260.4) 0.555 425.4 (231.9) 0.117 380.0 (169.8) 0.151 | 1.1 [-1.1-3.2] -3.4 [-15.5-8.6] -8.7 [-19.7-2.4] 5.1 [-2.0-12.2] | -8.0-10.1 -55.0-48.0 -55.8-38.5 -25.3-35.5 |

Research has shown no significant differences and strong relationships (r ≥ 0.996) between isokinetic dynamometer and portable transducer measurements of peak force from isometric MVC assessments of the leg extensors and flexors. However, other research comparing isometric leg extension and flexion peak force or torque measurements between isokinetic dynamometers and portable transducers has demonstrated significant differences and smaller relationships (r ≤ 0.884) between devices. The discrepancies between these findings and those reported by the present study may be a result of differences in isometric testing procedures and setups, the age ranges of the subjects, and/or the type of portable transducer used to assess isometric force or torque. The present study assessed isometric torque measures using the dynamo, which included a load cell and microcomputer, whereas the previous research evaluated isometric force or torque measures using commercially available hand-held transducers with built-in strain gauges and output displays. It has been reported that the end pieces of many hand-held transducers are not adequately padded to prevent discomfort during isometric testing. Excessive pain or discomfort during testing can cause nonmaximal efforts by the subject, which could lead to substantial decreases in force production and increases in transducer measurement error. Alternatively, the dynamo proposed in the present study uses a load cell with mounting holes on either end of the device. These mounting holes provide for easy attachment of the load cell to comfortable, well-padded equipment (i.e., isokinetic dynamometer, resistance training machine, therapy boot, treatment table or chair, etc.), which may be a necessary component for acquiring valid and reliable isometric torque data.

Previous research comparing an isokinetic dynamometer and a portable device showed that the portable device significantly underestimated isometric leg extension RTD characteristics, including RTD100, when compared to an isokinetic dynamometer, which is inconsistent with the present findings. However, unlike the present study which obtained RTD values from the dynamo with the load cell attached to the isokinetic dynamometer, the previous research measured RTD values from a hand-held transducer that was attached to a treatment table using a restraining belt. Moreover, the hand-held transducer used in the previous research recorded force at a sampling frequency of 100 Hz instead of 1,000 or 2,000 Hz which represents the sampling frequencies of choice for most isokinetic dynamometer assessments. It has been suggested that the force or torque signal from a transducer should be sampled at a high frequency of at least 1,000 Hz to acquire accurate measurements of RTD. Although further research is needed to compare RTD characteristics derived from torque signals sampled at different frequencies, it is possible that recording force or torque data from a portable transducer at 1,000 Hz, which was the sampling frequency used by the dynamo in the present study, may yield RTD values that are statistically similar and more closely related to those of an isokinetic dynamometer.

Isokinetic dynamometers and hand-held transducers offer a useful means for quantifying real-time measurements of isometric peak force or torque; however, they do not provide real-time calculation of isometric RTD. Such calculation requires a fully functioning computer with additional hardware and software and extensive post-testing analysis. In contrast, the dynamo used in the present study automatically calculates and displays early, late, and maximum RTD parameters immediately after an isometric contraction. Given the accuracy and speed with which it can analyze isometric torque, the Dynamo may be highly desirable in research or clinical situations where rapid data analysis is required for immediate RTD results. Real-time RTD calculation with the Dynamo may create unique opportunities for both the patient and the practitioner to interact in real-time with RTD data during patient examination and treatment. Researchers and practitioners who want to provide patients with RTD results immediately before and after training or rehabilitation could benefit from the Dynamo’s real-time quantification and display of RTD variables. Immediate knowledge of RTD results also enables patients to compare their own RTD data with past performance and that of other patients, which may serve as a motivational tool to help them exert maximal effort during testing.

### Practical Applications

Mean values of 162 - 218 Nm and 526 - 801 Nm·s⁻¹ for the leg extensors and 72 - 115 Nm and 242 - 365 Nm·s⁻¹ for the leg flexors have been reported previously in young healthy adults for isometric PT and RTD200 measurements. These results are similar to the mean dynamo PT and RTD200 values observed for the leg extensors (180 Nm and 646 Nm·s-¹) and flexors (102 Nm and 380 Nm·s⁻¹) in the present study. Of the 20 subjects in this study, two male subjects produced dynamo isometric leg extension PT (332 - 337 Nm) and RTD200 (1194 - 1249 Nm·s⁻¹) values that were within the range of isometric leg extension PT (255 -485 Nm) and RTD200 (1070 - 2120 Nm·s⁻¹) values produced by elite collegiate American football players. These findings provide support that the Dynamo may be able to effectively calculate the lower-body maximal and rapid torque measurements of athletes, including highly trained football players with superior levels of muscle strength. Given the potential importance of isometric RTD to explosive performances, coaches and other practitioners may want to consider using dynamo measurements of RTD in their current test battery. These measurements may provide coaches with an additional evaluation tool to help in identifying athletes with high rapid strength values and possibly overall athletic ability. Further research examining the utility of the dynamo as an effective portable measurement device to predict the explosive performance capacities of athletes is needed to test this hypothesis.

Finally, research has reported that isometric RTD of the lower-body musculature is an important characteristic relevant to functional performances (i.e., timed up and go, sit-to-stand, gait speed, etc.) in older adults and other clinical populations. Because the Dynamo calculates and displays real-time measurements of RTD, these measurements may be useful in the identification and early detection of older adults who are at a high risk for functional performance deficits. Future studies investigating the efficacy of dynamo RTD measurements to differentiate between older adults of varying health statuses and functional performance abilities are needed to further examine these findings.

### Conclusions

This investigation revealed no systematic variability across sessions and the ICC and SEM% values ranged from 0.935 to 0.984 and 4.93 to 14.46%, respectively, for isometric leg extension and flexion PT and RTD characteristics for the isokinetic dynamometer and dynamo. In addition, significant positive relationships were observed between the isokinetic dynamometer and dynamo for all PT and RTD characteristics, and there were no significant differences between devices for these variables. Taken together, these findings provide support that the dynamo is a valid and reliable device for measuring isometric PT and RTD of the lower-body musculature. RTD characteristics as assessed from an isometric leg extension or flexion MVC have functional significance in fast and forceful movements and thus, may be of vital importance for determining explosive performance abilities. The dynamo torque analyzer proposed in the present study may provide researchers and practitioners with a relatively accurate, time- and cost-effective assessment tool capable of enhancing the practicality and utility of these measurements when examining the explosive performance capacities of athletes in both laboratory and field-based settings.

### Use of Dynamo PT and RTD to Evaluate Physical Function

### Subjects

Twenty older women were recruited to participate in the present study (demographics are listed in Table 4).

TABLE 4

Mean ± SD values for age, height, body mass, and six-minute walk distance.

| Variable | Higher Functioning | Lower Functioning |
|---|---|---|
| Age (years) | 66.6 ± 4.20 | 67.60 ± 4.43 |
| Height (cm) | 159.45 ± 2.91 | 159.32 ± 8.03 |
| Body Mass (kg) | 66.78 ± 6.99 | 68.29 ± 6.76 |
| Walk Distance (m) | 627.43 ± 49.34* | 492.56 ± 42.33 |

*Significant difference (P<0.050) between the higher and lower functioning groups

Subjects were recruited from the local community via advertisements, flyers, and word of mouth. To be eligible for the study, subjects had to walk without an assistive device and have no orthopedic limitations or problems with the ankle, knee, or hip joints. No subject reported any current or ongoing neuromuscular diseases or musculoskeletal injuries to these areas. Consequently, all were eligible to participate. The subjects were classified into a “higher functioning” or “lower functioning” group according to their distance covered during a six-minute walk test. A distance of 550 m during the six-minute walk test has been reported and used as a cutoff for distinguishing between functional status in older adults and other clinical populations. Based on this criterion, of the 20 subjects in the present study, 10 were identified as higher functioning (≥550 m) and 10 were identified as lower functioning (<550 m). This study was approved by the university’s institutional review board for human subject research, and each subject signed and completed an informed consent document and health history questionnaire.

### Experimental Design

This study used a cross-sectional research design to investigate the ability of isometric knee extension PT and RTD characteristics to distinguish between functional differences in older women according to their performance on a six-minute walk test. Subjects reported to the laboratory for a single visit where they signed and completed the informed consent document and health history questionnaire. After completing the paperwork, the subjects performed three isometric knee extension MVCs followed by a six-minute walk test.

### Isometric Knee Extension

Isometric knee extension MVCs were performed on the right leg using a dynamo, for example dynamo 100, (FIG. 4). The dynamo included a microcomputer and a load cell (200 kg capacity) that was attached to an ankle pad of a lever arm fastened around the subject’s lower leg. The lever arm was connected to an input axis of an isokinetic dynamometer (Biodex System 3, Biodex Medical Systems Inc., Shirley, NY) which was aligned with the axis of rotation of the knee joint. For each MVC, subjects sat on the dynamometer chair in an upright position with restraining straps placed over the shoulders, waist, and thigh. All MVCs were performed at a knee joint angle of 60 ° below the horizontal plane. Prior to the MVC assessments, subjects performed a standardized warm-up of three submaximal isometric knee extension muscle actions at approximately 75% of their perceived maximal effort. Following the submaximal contractions, each subject performed three isometric knee extension MVCs with one minute of recovery between each trial. For all isometric MVCs, subjects were verbally instructed to push “as hard and fast as possible” for a total of 3-4 seconds and strong verbal encouragement was given throughout the duration of the contraction.

### Data Processing

During each MVC, the scaled force signal from the load cell was sampled, interpolated to 1,000 Hz, and processed automatically by the dynamo. A torque signal (Nm) was derived by multiplying the force signal (N) from the load cell by the limb length (m) for each subject. Limb length was measured as the distance from the lateral knee joint to the ankle (positioned over the load cell) and was entered into the dynamo’s microcomputer prior to the MVC assessments. The torque signal was gravity corrected and low-pass filtered with a zero-phase lag, fourth-order Butterworth filter at a cutoff frequency of 150 Hz. All subsequent analyses were conducted on the filtered and gravity-corrected torque signal.

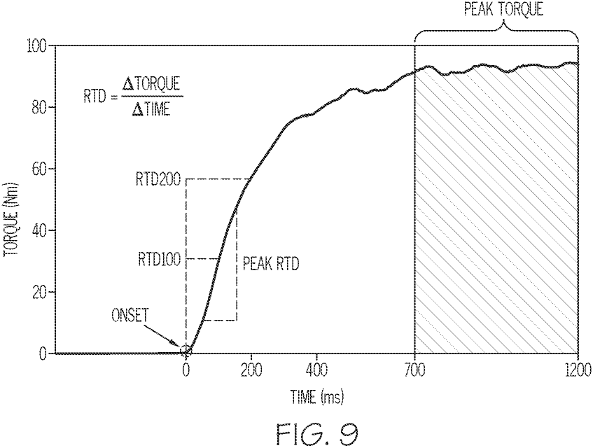

Isometric PT was calculated as the highest mean 500 ms epoch (FIG. 9). RTD was calculated as the linear slope of the torque signal (Δtorque/Δtime) at early and late time intervals of 0-100 (RTD100) and 0-200 (RTD200) ms from contraction onset (FIG. 9). Maximum RTD (Peak RTD) was calculated as the highest slope value for any 100 ms epoch that occurred over the initial 200 ms of the torque signal (FIG. 9). The contraction onset for the dynamo was set at 1.0 Nm. Isometric PT, Peak RTD, RTD100, and RTD200 were calculated and displayed by the dynamo at the conclusion of each trial and were normalized to body mass.

Unsteady baseline torque resulting from either pre-tension or countermovement can adversely influence RTD. A unique feature of the dynamo is its ability to detect unsteady baseline torque. The dynamo’s microcomputer evaluates unsteady baseline torque by computing the baseline slope prior to contraction onset. If unsteady baseline torque was detected prior to contraction onset, a warning was displayed by the microcomputer at the end of the trial. Contractions with unsteady baseline torques as indicated by the dynamo were always discarded, and additional MVCs were performed until three knee extension contractions presented acceptable data. Of the three MVCs performed, the MVC with the highest PT value was selected for further analysis.

### Six-Minute Walk Test

The six-minute walk test was performed in accordance with the procedures described by the American Thoracic Society, in which subjects walked back and forth between two markers set 30 m apart (60 m per lap). Subjects were instructed to walk as fast as possible, without running, at a pace that they could maintain for six minutes. No subjects had to stop or rest during the test. Only one six-minute walk test was performed, and no warm-up period before the test was permitted. The primary investigator timed the walk with a stopwatch and used a mechanical counter to count the number of laps completed. Standardized words of encouragement (i.e., “you are doing well,” “keep up the good work”) were given at 30-s intervals, and subjects were informed of the remaining time left at each minute mark. The total distance covered (m) at the end of six minutes was recorded and used to separate the subjects into higher (≥550 m) or lower (<550 m) functioning groups.

### Reliability

Reliability analysis for the distance covered during the six-minute walk test and dynamo PT and RTD variables was performed on a subset of the subjects on two nonconsecutive days. The intraclass correlation coefficient and standard error of measurement expressed as a percentage of the mean were 0.83 and 4.6% for the six-minute walk distance, 0.91 and 6.7% for PT, 0.95 and 8.9% for Peak RTD, 0.93 and 13.9% for RTD100, and 0.92 and 9.8% for RTD200, respectively. In addition, there were no systematic differences (P > 0.050) between testing days for any of the variables.

### Statistical Analyses

Independent samples t-tests were used to compare demographic characteristics, six-minute walk distance, PT, and RTD variables between the higher and lower functioning groups. Cohen’s d effect sizes and percent differences (%Δ) were calculated for each between-group comparison. Pearson correlation coefficients (r) were calculated to examine the relationships between six-minute walk distance and PT and RTD variables. Multiple regression analysis (stepwise model) was used to determine which variables were the best predictors of six-minute walk distance. Discriminant analysis was used to establish critical PT and RTD thresholds for identifying functional group membership. All statistical analyses were performed using IBM SPSS Statistics Version 26.0 (SPSS Inc., Chicago, IL), and an alpha level of P ≤ 0.050 was used to determine statistical significance.

### Results

Mean and standard deviation (SD) values for demographic characteristics and six-minute walk distance are presented in Table 4. There were no significant differences between the higher and lower functioning groups for age (P = 0.610, d = 0.24, %Δ = 1.5%), height (P = 0.963, d = 0.02, %Δ = 0.1%), or body mass (P = 0.629, d = 0.22, %Δ = 2.3%). Six-minute walk distance was significantly greater for the higher compared to the lower functioning groups (P < 0.001, d = 1.64, %Δ = 21.5%). Table 5 shows the means, SDs, P values, Cohen’s d effect sizes, and percent differences between groups for isometric PT and RTD variables.

TABLE 5

Means ± SDs, P values, Cohen’s d effect sizes, and percent differences (%Δ) between groups for isometric peak torque and rate of torque development variables.

| Variable | Higher Functioning | Lower Functioning | P value | d | %Δ |
|---|---|---|---|---|---|
| Peak Torque (Nm kg⁻¹) | 1.82 ± 0.30* | 1.45 ± 0.28 | 0.011 | 1.08 | 20.3% |
| Peak RTD (Nm s⁻¹ kg⁻¹) | 12.35 ± 2.43* | 6.32 ± 1.71 | <0.001 | 1.63 | 48.8% |
| RTD100 (Nm s⁻¹ kg-¹) | 11.88 ± 2.49* | 5.36 ± 1.97 | <0.001 | 1.63 | 54.9% |
| RTD200 (Nm s⁻¹ kg-¹) | 7.69 ± 1.20* | 4.62 ± 1.03 | <0.001 | 1.6 | 39.9% |

*Significant difference (P ≤ 0.050) between the higher and lower functioning groups

The higher functioning group exhibited significantly greater PT, Peak RTD, RTD100, and RTD200 compared to the lower functioning group (P ≤ 0.011, d ≥1.08), with larger differences occurring for RTD characteristics (%Δ = 39.9-54.9%) than PT (%Δ = 20.3%).

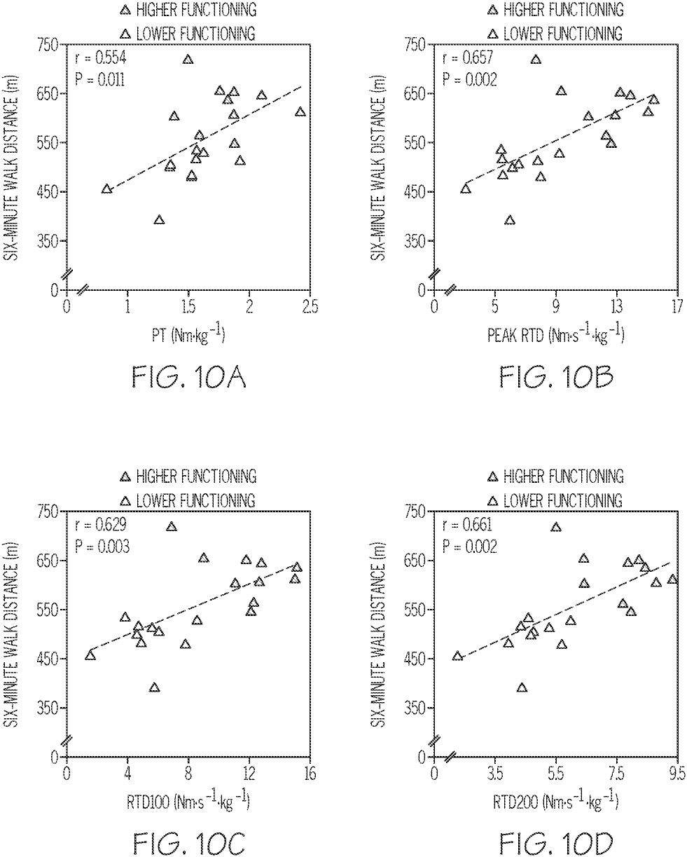

Significant positive relationships were observed between six-minute walk distance and PT (r = 0.554, P = 0.011, FIG. 10A), Peak RTD (r = 0.657, P = 0.002, FIG. 10B), RTD100 (r = 0.629, P = 0.003, FIG. 10C), and RTD200 (r = 0.661, P = 0.002, FIG. 10D). For the multiple regression analysis, isometric PT, Peak RTD, RTD100, and RTD200 were entered as predictor variables into the stepwise model. The model found RTD200 to be the single best predictor of the distance covered during the six-minute walk test (R2 = 0.437, P = 0.002).

Discriminant analysis revealed thresholds of 1.58 Nm·kg⁻¹ for PT and 9.27, 8.80, and 6.22 Nm·s⁻¹·kg⁻¹ for Peak RTD, RTD100, and RTD200, respectively. The thresholds for the RTD variables demonstrated excellent sensitivity (100%) and specificity (90%) for identifying functional group membership. All discriminant analysis statistics for PT and RTD variables are shown in Table 6.

TABLE 6

Discriminant analysis statistics for identifying functional group membership.

| Variable | Threshold | Sensitivity % | Specificity % |
|---|---|---|---|
| Peak Torque (Nm kg-¹) | 1.58 | 80 | 80 |
| Peak RTD (Nm s⁻¹ kg-¹) | 9.27 | 100 | 90 |
| RTD100 (Nm s⁻¹ kg-¹) | 8.8 | 100 | 90 |
| RTD200 (Nm s⁻¹ kg-¹) | 6.22 | 100 | 90 |

### Discussion

The primary findings of this study revealed that isometric knee extension PT and RTD characteristics were significantly greater in the higher compared to the lower functioning groups, with larger differences occurring for RTD than PT (Table 5). There were significant positive relationships between six-minute walk distance and PT and RTD characteristics (FIGS. 8A-8D). Multiple regression analysis indicated that RTD200 was the single best predictor of the distance covered during the six-minute walk test. Moreover, the thresholds for the RTD variables in the discriminant analysis demonstrated excellent sensitivity and specificity for identifying functional group membership (Table 6).

The greater PT and RTD characteristics observed in the present study for the higher compared to the lower functioning groups demonstrated the effectiveness of these variables at distinguishing between functional status in healthy older women. Studies have reported similar findings regarding the efficacy of isometric PT and RTD variables to discriminate between older adults of different functional performance abilities; for example, it has been shown previously that older adults who were able to walk 10 m at a faster speed were also able to produce greater isometric PT and RTD of the knee extensors than age-matched slower individuals. It was hypothesized that because the faster walkers exhibited greater PT and RTD values than the slower walkers, maximal and rapid strength may be important determinants of 10-m gait speed in older populations. Similar to gait speed, the six-minute walk test, despite being a longer duration event, still requires a certain level of muscle strength and power and, therefore, may also be influenced by maximal and rapid strength characteristics. Evidence suggests that older adults with lower maximal and rapid strength use a greater percentage of their force-generating capacity to successfully ambulate over ground. Operating at a greater capacity contributes to the early onset of fatigue, which in turn, may reduce one’s ability to walk faster and cover a longer distance within a specific duration of time. The results of the present study add support to the importance of maximal and rapid strength in regard to the distance covered during a six-minute walk test, given that this was the parameter used to separate subjects into higher or lower functioning groups. Because walking places a high demand on the lower extremity muscles, including those surrounding the knee joint, it is possible that knee extension PT and RTD characteristics may be effective parameters at determining walking performance abilities in older adults.

A key finding of this study was that the differences in RTD characteristics (39.9-54.9%) between the higher and lower functioning groups were larger than the difference in PT (20.3%). This finding is consistent with the findings of previous research, which showed larger differences for knee extension RTD (36.2%) than PT (14.5%) between older women with slow and fast gait speeds. Collectively, these findings highlight the importance of rapid strength and suggest that RTD may be a better variable than PT at distinguishing between older women of different walking performance abilities. Many locomotor movement tasks, including walking, involve rapid, repetitive muscle actions of the knee extensors. During normal walking, knee extensor muscle activity begins in terminal swing and rapidly increases to peak amplitude early in the loading response, requiring force to be generated within approximately 150 ms. Because the time required to achieve maximal force is typically greater than 300 ms, rapid strength characteristics (0-200 ms) of the knee extensors may be more functionally relevant than maximal strength for walking-related tasks in older adults. Therefore, the possibility of greater functional relevance between rapid strength and walking performance ability may explain why larger differences in RTD characteristics were observed between the higher and lower functioning groups in the present study.

Poor performance on the six-minute walk test may be due to low rapid strength capacities. Studies have suggested that the inability to produce torque rapidly is a limiting factor in the performances of walking-related tasks and that lower rapid strength may lead to a decreased ability to walk faster and/or cover long distances in a timely manner. The present findings support these hypotheses, given the significant positive relationships (r = 0.629-0.661, FIGS. 8B-8D) between knee extension RTD characteristics and six-minute walk distance in older women. Similarly, other research demonstrated a significant relationship in older women between RTD of the knee extensor muscles and maximal gait speed (r = 0.602). Taken together, these findings suggest that rapid strength of the knee extensors may be an important characteristic relevant to walking performance abilities in older adults. It is noteworthy that the present findings showed a significant relationship between six-minute walk distance and PT (r = 0.554, FIG. 8A). Although these findings highlight the potential for PT to predict performance on the six-minute walk test, multiple regression analysis revealed that the single best predictor of six-minute walk distance was RTD200 (R² = 0.437). The superior predictive capacity of RTD200 versus PT may be due to the aforementioned greater functional relevance of rapid strength as it pertains to the fast and forceful muscle actions required to perform important walking-related tasks. Research has shown that RTD of the knee extensors was a more effective variable than PT at predicting 10-m gait speed in older adults. The results of our study extend these findings by demonstrating that knee extension RTD may also be a better predictor than PT of the distance covered during a six-minute walk test.

Although the present findings and those of previous research provide support that RTD is an important characteristic relevant to walking performance abilities in older adults, it is acknowledged that other variables may also play a role in one’s ability to successfully ambulate over ground. Investigations have reported that walking performance abilities in older adults may be related to maximum oxygen uptake, standing balance, and joint range of motion at the hip and knee. Further research is needed to determine the variables that are most important for explaining the variance associated with walking performance abilities in older populations.

The thresholds for the RTD variables in our discriminant analysis showed 100% sensitivity (ability to correctly identify those who were lower functioning) and 90% specificity (ability to correctly identify those who were higher functioning) (Table 6). These sensitivity and specificity statistics were higher than those for PT (80%), which supports our previous assertion that the ability to generate torque rapidly may be a better discriminator of functional performance ability in older adults than maximal strength. In this study, isometric RTD values were substantially lower in the older women who were unable to walk 550 m during the six-minute walk test. Because ~550 m is considered the maximal walking distance required for successful community ambulation, the inability to cover such a distance in a timely manner may contribute to a lack of functional independence and confidence in community level participation. Thus, in light of this and given the high sensitivity and specificity observed for the RTD thresholds in the discriminant analysis, it is possible that isometric RTD may be an effective measure at identifying older adults who are at risk for functional decline. In the present study, isometric RTD characteristics were considerably smaller in the lower functioning group. Research suggests that differences in RTD may be attributed to several factors including changes in connective tissue stiffness, type II fiber area, and muscle activation characteristics. Because age-related changes in RTD are believed to be influenced by factors that include muscle size and strength, the smaller RTD values we observed in the lower functioning group may be a result of impairments in these physiological mechanisms. Consequently, training programs aimed at increasing the size and strength of the lower-body musculature may be beneficial for improving RTD as well as mobility in older adults with low functional status. Future studies investigating the effects of muscle strength and hypertrophy training on rapid strength characteristics and walking ability in lower-functioning older adults are needed to further examine these findings.

Commercial devices, such as isokinetic dynamometers and hand-held transducers, are commonly used to measure isometric PT and RTD characteristics. To calculate RTD with these devices, offline analysis of the torque signal using data processing software is required. Because analyzing the torque signal offline can be a difficult and time-consuming task, this method of RTD calculation may not be feasible in certain research or clinical situations where rapid data analysis is required for immediate RTD results. In contrast, the dynamo described herein automatically calculates and displays in real time early, late, and maximum RTD parameters immediately after an isometric contraction. The present findings provide support that RTD measurements from the dynamo may be particularly useful at distinguishing between older women of different functional status. Given the potential importance of rapid strength to locomotor-related movement tasks, physical therapists and other practitioners may want to consider using dynamo measurements of RTD in their current test battery. These measurements may provide practitioners with an additional evaluation tool to help in identifying older adults who are at a high risk for functional performance deficits.

In summary, our findings showed that the higher functioning women exhibited greater isometric PT and RTD characteristics of the knee extensors than the lower functioning women, with larger differences occurring between groups for RTD than PT. These findings suggest that knee extensor muscle strength, and in particular RTD, may be an effective measure at distinguishing between older women of different functional status. An interesting finding of this study was the significant positive relationships between six-minute walk distance and isometric RTD characteristics. Because the best predictor of six-minute walk distance was RTD200, the present findings provide support that the ability to generate torque rapidly (0-200 ms) may play an important role in the distance covered during a six-minute walk test in older adults. The thresholds for the RTD variables in the discriminant analysis demonstrated high sensitivity and specificity, and, therefore, may be used as indices to identify older adults with low functional performance abilities. A novel aspect of the present study was the utility of the dynamo. The ability of the dynamo to provide real-time measurements of isometric RTD that are highly discriminatory of functional differences in the elderly may make it an attractive evaluation tool for purposes of assessing the lower-body performance capacities of older adults in both laboratory and field-based settings.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of analyzing a subject using a dynamo torque analyzer, the method comprising:
measuring, via the dynamo torque analyzer, a force produced by the subject during an isometric contraction;
collecting, via the dynamo torque analyzer, data associated with the force produced by the subject during an isometric contraction; and
calculating, via the dynamo torque analyzer, a torque value using the data,
wherein the dynamo torque analyzer comprises:
a microcomputer comprising a CPU and memory; and
a load cell in communication with the microcomputer.

2. The method of claim 1, further comprising, after the collecting, processing, via the dynamo torque analyzer, the data associated with the isometric contraction.

3. The method of claim 2, wherein the processing comprises processing the data associated with the isometric contraction with a dual-pass Butterworth filter.

4. The method of claim 3, wherein a cutoff frequency ranging between 5 Hz and 200 Hz is used.

5. The method of claim 2, wherein the processing comprises converting a voltage output from the load cell to newtons.

6. The method of claim 5, wherein a set of calibration parameters includes a slope (m) and intercept (b) value that is used to calibrate the dynamo torque analyzer.

7. The method of claim 2, wherein the processing comprises using a limb length of the subject to convert newtons to newton-meters.

8. The method of claim 2, wherein the processing comprises correcting the data for gravity.

9. The method of claim 2, wherein the processing comprises interpolating the data to 1,000 Hz.

10. The method of claim 1, wherein the torque value is a peak torque.

11. The method of claim 1, wherein the torque value is a rate of torque development (RTD).

12. The method of claim 1, wherein the torque value is a RTD100 calculated as a linear slope of a torque signal of the data at a time interval of 0-100 ms.

13. The method of claim 1, wherein the torque value is a RTD200 calculated as a linear slope of a torque signal of the data at a time interval of 0-200 ms.

14. The method of claim 1, wherein the torque value is a peak RTD calculated as the highest slope value for any 100 ms epoch that occurs over an initial 200 ms of a torque signal of the data.

15. The method of claim 1, further comprising performing, by the dynamo torque analyzer, a baseline check to determine if a countermovement occurred.

16. The method of claim 15, wherein, responsive to a determination by the dynamo torque analyzer that a countermovement has occurred, providing an indication that a new trial needs to be performed.

17. The method of claim 1, further comprising setting, by the dynamo torque analyzer, a contraction onset to determine a start of the isometric contraction.

18. The method of claim 1, wherein the calculating uses a limb length of the subject.

19. A dynamo torque analyzer comprising:
a microcomputer comprising a CPU and memory;
a display in communication with the microcomputer;
a load cell in communication with the microcomputer; and
a power supply coupled to the microcomputer.

20. The dynamo torque analyzer of claim 19, wherein the load cell comprises first and second mounting points.

21. The dynamo torque analyzer of claim 20, further comprising an ankle cuff, pad, or boot attached to the first mounting point.

22. The dynamo torque analyzer of claim 19, wherein the load cell is an S-type load cell.

23. The dynamo torque analyzer of claim 19, further comprising an amplifier and an analog to digital converter that are coupled between the load cell and the microcomputer.

24. The dynamo torque analyzer of claim 19, further comprising a battery coupled to the power supply.
